(19) United States
(12) Patent Application Publication
(10) Pub. No.: US 2012/0065498 A1
(43) Pub. Date: Mar. 15, 2012

(54) APPARATUS AND METHOD FOR MINIMALLY INVASIVE THERAPY OF MITRAL REGURGITATION

(76) Inventors: Thomas Redel, Poxdorf (DE); Michael Maschke, Lonnerstadt (DE); Frederik Bender, Erlangen (DE)

(21) Appl. No.: 12/881,925

(22) Filed: Sep. 14, 2010

(51) Int. Cl. A61B 5/05; A61B 8/00

(52) U.S. Cl. 600/424; 600/439

(57) ABSTRACT

A system and method of treatment of mitral valve insufficiency using an implantable medical device is described. The system uses a C-arm X-ray device configured to produce computed-tomographic (CT)-like images, and to superimpose the CT-like images on a fluoroscopic image taken with the same X-ray device as a part of the treatment procedure. The fluoroscopic images are used to guide a catheter in the patient so as to place the medical device in a proper position. The efficacy of the procedure may be assessed using a non-invasive or minimally invasive acoustic imaging technique.

### TECHNICAL FIELD

The application relates to a system and method for medical therapy using computed tomographic and fluoroscopic images for treatment device guidance.

### BACKGROUND

Mitral valve insufficiency (MI), also called mitral regurgitation (MR), is a cardiac valve defect that frequently occurs in humans. The syndrome involves an inability to close, or a “leakiness” of the mitral valve of the heart which, during the ejection phase (systole), leads to a reverse flow of blood from the left ventricle to the left atrium. During the systole, blood flows through the mitral valve from the left ventricle into the left atrium, which is undesirable.

The mitral valve functions as a valve between the left atrium and the left ventricle of the heart. In the filling phase of the ventricle (diastole), the mitral valve opens and thus enables the inflow of blood from the atrium. At the onset of the ejection phase (systole), the suddenly rising pressure in the ventricle leads to closure of the valve and thus to a “sealing off” of the atrium. In this way, in the atrium the pressure of only about 8 mmHg, while in the ventricle the systolic pressure of approximately 120 mmHg drives the blood into the main artery (aorta).

In severe mitral insufficiency, conversely, the regurgitation opening is more than 40 mm², and the regurgitation volume is more than 60 ml, which can lead to severe and sometimes life-threatening consequences.

In the acute stage of mitral valve insufficiency, with a normal size of the left ventricle and left atrium, a considerable increase in the pressure in the atrium occurs, and thus in the pulmonary veins as well. This pressure can be as high as 100 mmHG, which, even when the vessels of the lung are in their normal condition, leads to immediate lung edema. Moreover, the predominantly reverse flow of blood can also cause an inadequate ejection output from the heart into the aorta and thus to inadequate profusion of all the organs.

Once the acute stage of the syndrome has been withstood, or if the mitral insufficiency develops over a longer period of time, the result is a series of chromic adaptation processes in the heart and in the vessels of the lung. First, the persistent strain of pressure and volume on the atrium causes enlargement thereof, and the atrium volume can often increase to three to four times the normal values within months or years. This dilation over the course of time also lessens the pressure-increasing effect of the regurgitation volume in the lung circulation. In addition, the volume strain also causes an enlargement of the left ventricle which, with every heartbeat must, in addition to the actual quantity of blood needed, also pump the regurgitation volume. This dilation can on the one hand by way of the Frank-Starling mechanism also increase the stroke volume but on the other leads to a vicious cycle, if with the expansion of the ventricle the geometry of the mitral valve is also disturbed, and in this way its insufficiency is increased still further.

Typically, mitral insufficiencies are classified by degrees of severity; at present, usually three stages (slight, medium-severe, and severe), and sometimes even four (grade I to grade IV) are distinguished.

Acquired mitral insufficiency from, for example, cleavage of the anterior mitral cusp, and dramatic mitral insufficiency as a consequence of rheumatic fever are by now rare in industrialized countries, but in developing countries the causes continue to occur frequently.

The problems of most importance at present are post-infarction mitral insufficiency after a heart infarct, ischemic mitral insufficiency caused by problems with circulation to the heart muscle, relative mitral insufficiency as a consequence of an enlargement of the left ventricle, and mitral insufficiency in prolapse syndrome in conjunction with a usually congenital mitral valve prolapse.

Bacterial as well as nonbacterial endocarditis can also lead to destruction or shrinkage of the mitral valve from scarring of the valve tissue and thus lead to mitral insufficiency.

Congenital mitral valve insufficiency is either observed in isolated cases as a consequence of cleavage of the front (anterior) mitral cusp or a defective position (dysplasia) of the mitral cusp or more frequently as a so-called “complex valve defect” in conjunction with other cardiac defects, such as transposition of the major arteries, a corrected transposition, a double outlet right ventricle, atrial septum defect, or ventricle septum defect.

The less severe forms of the defect are not observed by the person affected. The typical symptoms of severe mitral insufficiency are exhaustion after little effort and shortness of breath (dispnea). Cardiac arrhythmias, which occur more often in mitral insufficiency, can make themselves felt in the form of missing beats or heart palpitation.

The most important and indicative finding in physical examination is a high-frequency characteristic systolic heart sound, which can usually be heard at its loudest above the apex of the heart and into the left armpit. In left ventricular dilation, sometimes a shifted apex beat, in the case of pulmonary congestion pulmonary wheezing, and in secondary right heart failure, congestion of the veins of the neck and edema can be found.

For assessing mitral insufficiency, besides the physical examination an ultrasound examination of the heart may be performed; in cases of doubt, ultrasound examination can be in the form of trans-esophageal echocardiography (TEE). Other examination methods may be needed only in special cases or before a planned operation, for excluding accompanying illnesses.

In slight mitral insufficiency, no therapy may be considered necessary. However, normal blood pressure values in the patient should be maintained, since high blood pressure increases the pressure difference between the left ventricle and the atrium and thus increases the regurgitation volume and the pressure strain on the atrium.

In severe mitral insufficiency with signs of heart failure, treatment is oriented to the principles of heart failure treatment. Whether long-term medical treatment with ACE blockers improves the prognosis even in asymptomatic patients without heart failure is still disputed. If arrhythmias are simultaneously present, the use of antiarrhythmic medications may be necessary.

Depending on the size of the left atrium, the use of anticoagulents such as Phenprocoumon or Warfarin may be necessary for prophylaxis of thrombosis inside the (enlarged) left atrium.

In acute severe mitral insufficiency, the treatment must usually be done in the intensive care unit (ICU). The goal of the medical therapy is to reduce the regurgitation volume, on the one hand, to increase the forward flow and, on the other hand, to reduce the pulmonary congestion. In patients with normal blood pressure, this goal can be met with nitroprussid sodium; with low blood pressure, the additional administration of DOBUTAMIN may be appropriate. Such patients also often benefit from the application of the intra-aortal balloon pump, which can contribute to stabilization in the preparatory phase of the requisite valve surgery.

In all patients with severe mitral insufficiency, the indication for a cardiac valve operation may be investigated. In this surgery, either the flap is reconstructed, or an artificial heart valve is inserted.

In principle, valve reconstruction should be preferred since it leads to a lesser impairment in cardiac function, and when a sinus rhythm is obtained it requires no long-term blood thinning. However, especially when the valve cusps are severely shrunken, calcified, or even destroyed, reconstruction is not possible, and only valve replacement can be considered. The chances for valve reconstruction can be estimated reliably in advance with the aid of echocardiography; however, in individual cases, the necessity of an artificial valve does not become clear until during the operation.

Valve surgery is appropriate if symptoms unambiguously caused by mitral insufficiency cannot be eliminated by medications, as long as the pump function of the left ventricle is not too severely restricted (ejection fraction [EF]>30%).

In asymptomatic patients (without complaints) with severe mitral insufficiency, an operation may be recommended if there are indications of an overload on the heart. This is the case when there is restricted pump function (EF<60%) or considerable enlargement (end-systolic diameter [LVESD]>45 mm or LVESD Index >26 mm/m²) of the left ventricle, and also if there is evidence of pulmonary hypertension (systolic pulmonary artery pressure >50 mmHG (67 mbar) at rest or >60 mmHG (80 mbar) with exertion). If the mitral valve is reconstructable, the indication is within more generous limits, since the expected improvement through the surgery must be estimated as greater.

In cases of doubt, the determination of changes in pressure values and in the pump function of the heart with physical exertion (hemodynamic stress), can be helpful in the initial assessment of the indication for surgery.

Postoperatively, patients with a reconstructed valve are as a rule considered “heart healthy” after only a few weeks. If no other diseases are present, their exercise tolerance is not significantly restricted, and no particular cardiac-oriented treatment is necessary.

Patients with an artificial heart valve often require long-term anticoagulation with medication such as MARCUMAR. In such patients, the cardiac function under strain is sometimes measurably restricted, depending on the diameter of the prosthetic valve used. In otherwise normal cardiac function, this deviation, however, is so slight that in the everyday routine, no limitations can be felt by the patient.

Patients in whom the indication for surgery was determined on the basis of on current guidelines had an 8-year survival rate of 89%.

In recent years, research in the field of minimally invasive heart valve regurgitation has made progress. In this respect, the MitraClip (available from is known from Abbott Vascular, Menlo Park, Calif. (see also US patent application publications US 2005/0149014 and US 2006/0184203) may be used. With this clip, the leaflets of the mitral valve are clamped together using a minimally invasive intervention technique. This leads to better closure of the mitral valve and thus prevents the reverse flow of blood on the left from the ventricle to the left atrium and thus restores a satisfactory pumping capacity of the heart.

When describing a medical intervention technique, the terms “non-invasive”, “minimally invasive” and “invasive” may be used. Generally, the term non-invasive means the administering of a treatment or medication without introducing any treatment apparatus into the vascular system or opening a bodily cavity. Included in this definition is the administering of substances such as contrast agents using a needle or port into the vascular system. Minimally invasive means the administering of treatment or medication by introducing a device or apparatus through a small aperture in the skin into the vascular or related bodily structures. This includes the treatments known as percutaneous transluminal coronary angioplasty (PCTA), balloon angioplasty, stenting, other catheter-based techniques, and the like. Invasive techniques may include conventional surgery.

Minimally invasive treatment of mitral valve insufficiency with the MitraClip is currently being performed with the following devices:
C-arm x-ray system with fluoroscopy display;
TEE (trans esophageal echocardiography); and
a guide catheter.

The associated procedure (workflow) is:
1. Introducing the guide catheter in the right atrium using fluoroscopy
2. Puncturing the atrial septum using fluoroscopy and TEE for visualization and guidance;
3. Piercing the atrial septum and introducing the guide catheter into the left atrium using fluoroscopy and TEE;
4. Guiding the MitraClip catheter into the left atrium using fluoroscopy and TEE images;
5. Positioning the MitraClip using fluoroscopy and TEE above or at the cusps of the mitral valve;
6. Clamping the MitraClip to the cusps of the mitral valve;
7. Checking the tightness of the closed mitral valve and the perviousness of the open mitral valve using Doppler TEE (If the result is satisfactory, continue with Step 8; if not, return to Step 5);
8. Final clamping of the MitraClip;
9. Removal of all the catheters and tools using fluoroscopy; and
10. Concluding fluoroscopy to check whether all the catheters and tools have been removed.

A disadvantage of these devices and the associated workflow is that with fluoroscopy and TEE, the anatomy of the right and left atrium is not well visualized. Cardiology professionals can therefore not adequately assess how far the guide catheter and the MitraClip reach into the left atrium, resulting in a high risk of damage to the left atrial wall. Moreover, it is difficult with TEE to find an optimal puncture point of the atrial septum. If the puncture point is poorly chosen, that is, too high or too low, the MitraClip, because of the restricted mobility of the MitraClip catheter, cannot be positioned and mounted correctly on the mitral leaflets.

Another disadvantage of the present devices and workflow is that because of the location of the TEE in the esophagus, the patient must be anesthetized. This stresses the patient severely and, furthermore, involves considerable effort and expense.

### SUMMARY

A treatment suite for catheterization of a patient is described, including: a C-arm X-ray device configured to obtain digital image data suitable for producing computed-tomographic-like and fluoroscopic images; and a catheter device configured to emplace an implantable device in the patient. The computed-tomographic-image is superimposed on a fluoroscopic image during the use of the catheter device.

In another aspect, a method of treatment of a patient includes the steps of transporting the patient to the therapy unit; obtaining a computed-tomography (CT)-like image data of the patient region to be treated using a C-arm X-ray device; superimposing the CT-like image data on fluoroscopic data obtained during the process of catheter treatment of the patient, the fluoroscopic data being obtained by the same device as was used to acquire the CT-like image data; using the superimposed data to assist the guidance of the catheter so as to implant a therapeutic device in the patient; confirming the efficacy of the therapeutic device; and, finalizing the implantation of the therapeutic device if an evaluation of the performance device is determined to be satisfactory. The step of implanting the therapeutic device may be repeated if the initial results are not satisfactory.

### BRIEF DESCRIPTION OF THE DRAWINGS

FIG. 1 shows a block diagram of a treatment unit suitable for performing the method of an example;

FIG. 2 shows images of a CT like slice, a segmented organ derived from the CT-slice, the segmented organ data overlaid on a fluoroscopic image, and a fluoroscopic image marked up to assist in catheter guidance; and

FIG. 3 shows a workflow of an example of a method of using the treatment unit of FIG. 1.

### DESCRIPTION

Exemplary embodiments may be better understood with reference to the drawings, but these examples are not intended to be of a limiting nature. When a specific feature, structure, or characteristic is described in connection with an example, it will be understood that one skilled in the art may effect such feature, structure, or characteristic in connection with other examples, whether or not explicitly stated herein.

The examples of diseases, syndromes, conditions, and the like, and the types of treatment protocols described herein are by way of example, and are not meant to suggest that the method and apparatus is limited to those named, or the equivalents thereof. As the medical arts are continually advancing, the use of the methods and apparatus described herein may be expected to encompass a broader scope in the diagnosis and treatment of patients.

Embodiments of this invention may be implemented in hardware, firmware, software, or any combination thereof, and may include instructions stored on a machine-readable medium, which may be read and executed by one or more processors.

A “treatment unit”, or “therapy unit” and a method use thereof is described. Such a therapy unit may include at least some of the following equipment types integrated as a platform for performing diagnosis and treatment of a patient: an imaging modality, which may be a C-arm X-ray unit capable of producing radiographic data for computed tomography (CT)-like (3D) and fluoroscopic (2D) images; and, for example, one or more of: an image processor for at least one of soft tissue or contrast enhanced image data obtained by the imaging modality; an image fusion processor; a computer and interface for entering patient data; and, a data interface with a local area network or a wide area network. Suitable image and data display devices such as flat panel video displays may also be provided.

Instructions for implementing processes of the platform may be provided on computer-readable storage media or memories, such as a cache, buffer, RAM, removable media, including FLASH or magnetic memory devices, hard drives or other computer readable storage media. Computer readable storage media include various types of volatile and nonvolatile storage media. The functions, acts or tasks illustrated or described herein may be executed in response to one or more sets of instructions stored in or on computer readable storage media, as are known in the art or may be subsequently developed. The functions, acts or tasks to be performed may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

Communications between the devices, systems and applications may be by the use of either wired or wireless connections. Wireless communication may include, audio, radio, lightwave or other technique not requiring a physical connection between a transmitting device and a corresponding receiving device. While the communication may be described as being from a transmitter to a receiver, this does not exclude the reverse path, and a wireless communications device may include both transmitting and receiving functions. Such wireless communication may be performed by electronic devices capable of modulating data as signals on carrier waves for transmission, and receiving and demodulating such signals to recover the data. The devices may be compatible with an industry standard protocol such as IEEE 802.11b/g, or other protocols that exist, or may be developed.

The term “procedure”, “care path”, “clinical care plan”, “workflow” or similar terms as used herein refer to a method in a medical context that includes a number of worksteps associated with the diagnosis and/or treatment of an illness, or the like. For example, typical worksteps within a clinical care plan may include admission, screening, diagnostic testing, therapy, physical examinations, operations, ambulance care, out-patient care, in-patient care, specific syndrome-related care, and other steps. Worksteps may include a sequence of process steps, the use of specified treatment or diagnostic equipment; medical supplies, such as contrast agents, occluders, stents, clips, drugs, medical appliances; transportation of the patient; and, performing medical procedures requiring at least one of non-invasive, minimally invasive or invasive aspects, and the like.

Minimally invasive therapies, especially cardiological procedures, including diagnosis and treatment of cardiac syndromes may be performed by a therapy unit that may include an X-ray system with which CT-like soft-tissue images can be produced.

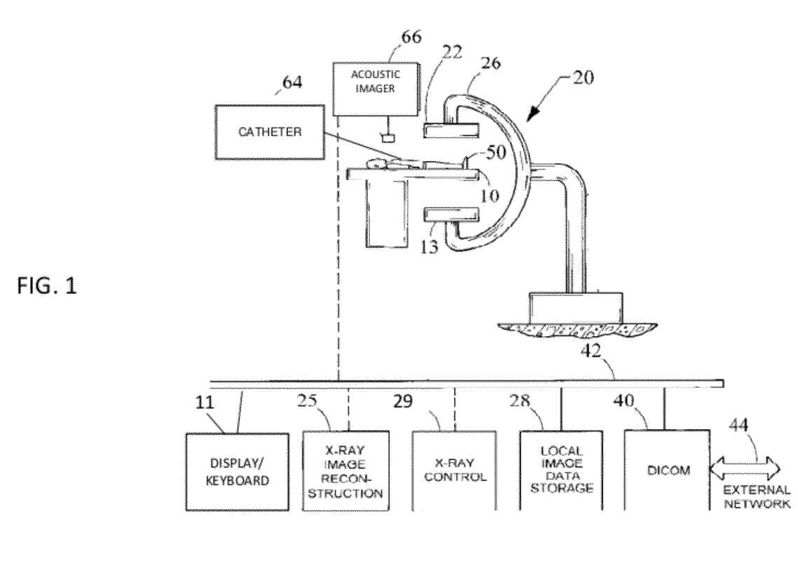

FIG. 1 shows a block diagram of an example of a therapy unit. Other embodiments of the therapy unit may include fewer than all of the devices, or functions, shown in FIG. 1. A C-arm X-ray device 20 may be used as an imaging modality to produce digital image data. The C-arm X-ray device 20 is rotated such that a sequence of projection-type X-ray images is obtained by a flat-panel X-ray detector 13 positioned on an opposite side of the patient 50 from the X-ray source 22, and the images are reconstructed by any computational technique of image data processing for realizing tomographic images. A patient support table may be used during the performance the method to support the patient 50 with respect to the C-arm X-ray device 20. A catheter system 64 would be used as part of the treatment portion of the method. The catheter system 66 may be used for various purposes, including the placement and adjustment of an implantable device such as the MitraClip used to treat a mitral valve insufficiency.

A C-arm X-ray configuration refers to C-shaped structural member 26 having an X-ray source 22 and an X-ray detector 13 typically mounted at or near the opposing open ends of the “C” such that a central ray of the X-radiation from the X-ray source is orthogonal to the surface of a facing X-ray detector. The space within the C-shape of the arm and the aperture to the “C” provides room maneuvering the patient 50 and other equipment and devices, such as the catheter 64 and acoustic imager 66, or for the physician to attend to the patient 50, with minimal interference from the X-ray support structure. This facilitates the use of other medical equipment while using the C-arm X-ray device 20 so as to support a minimally invasive interventional procedure with both fluoroscopic and CT-like images. An example of such a device is the AXIOM Artis dTA DynaCT (available from Siemens AG, Erlangen, Germany)

The C-arm can be mounted to permit rotational movement of the arm about two perpendicular axes in a spherical motion. The entire C-arm may also be translated in linear directions to facilitate positioning with respect to the patient, and enabling access for medical personnel and equipment.

Digital detector systems, which may be flat-panel real-time detectors for projection radiography are becoming commonplace in the clinical environment, and may be used to facilitate the rapid acquisition of data with the C-arm system. Such digital detectors provide high spatial resolution while having a high quantum efficiency. Apart from reducing the patient radiation dosage, such detectors may be highly linear and have sufficient resolution and dynamic range to be used in CT equivalent applications.

When the C-arm X-ray system uses a real-time X-ray detector, such as a flat-panel real-time X-ray detector, or any device having the same or similar capabilities, the C-arm may be rotated about the patient so that computed tomography-like (CT) images may be obtained. In such a use, image data acquisition may take approximately 10 seconds with C-arm rotation through approximately 200 degrees.

Additional, different, or fewer components may be provided. The devices and functions shown are representative, but not inclusive. The individual units, devices, or functions may communicate with each other over cables or in a wireless manner, and the use of dashed lines for some of the connections.

A display/keyboard 11 may be a notebook computer, tablet data entry and display device, or the like with which the demographic, history, diagnosis and/or therapy data of the patient can be recorded, called up and sent to and from the medical information management system of the hospital. The keyboard/display 11 may be provided with an interface for reading out the data from an HMO (health maintenance organization) or health insurance or card, and may be connected to the remainder of the treatment suite by a wireless connection. A user input device, such as a mouse, may be provided for manual input and control. In addition, the examination and therapy actions already performed may be documented in this computer device, including the medications administered or still to be administered. Some or all of the data may be forwarded to another entity for use in diagnosis, billing and administrative purposes, or further image processing and storage using known interfaces such as DICOM and SOARIAN, or special purpose or later developed data formatting and processing techniques. SOARIAN is a web-browser-based information management system for medical use, integrating clinical, financial, image, and patient management functions and facilitating retrieval and storage of patient information and the performance of analytic tasks (available from Siemens Medical Solutions Health Service Corporation, Malvern, Pa.).

In an aspect, the C-arm X-ray radiographic device 20 and the associated image processing 25 may produce angiographic or soft tissue computed-tomographic images comparable to, for example, CT equipment, while permitting more convenient access to the patient 50 for ancillary equipment such as the catheter 64, for treatment procedures. A separate processor 25 may be provided for this purpose, or the function may be combined with other processing functions, including the local image storage 28.

Images reconstructed from the X-ray data may be stored in a non-volatile (persistent) storage device 28 for further use. The X-ray device 20 and the image processing attendant thereto may be controlled by a separate controller 29 or the function may be consolidated with the user interface and display 11.

The various devices may communicate with a DICOM (Digital Communication in Medicine) system 40 and with external devices over a local area network 42, and a hospital or regional data base over a network interface 44.

The system and workflow for using contrast 3D C-arm images of the left atrium superimposed or fused with 2D fluoroscopy images of an X-ray system (3D-2D superposition) is described. By means of the 3D C-arm images, which may be CT-like images the anatomy of the atrium is shown in 3D. The image data may be segmented for visualization purposes, and soft-tissue images may also be produced.

Guiding the guide catheter and the MitraClip catheter pay be performed using live fluoroscopy images, in conjunction with the 3D image data obtained as part of the performance of the procedure. In addition, the optimal puncture point of the atrial septum can be marked in advance in the 3D data set and can also be superimposed on 2D fluoroscopy.

Registration of the 3D image data, segmented data with the fluoroscopic data is facilitated by using the same C-arm X-ray device for the both purposes while the patient is positioned for treatment.

All the structures relevant to the particular procedure, such as mitral valve annulus as well, can be superimposed from the 3D C-arm image. This includes characteristics extracted from the 3D C-arm image, such as segmentations (left atrium, for example) or drawn-in markings (for instance of the planned puncture point of the atrial septum and of the mitral valve annulus), regardless of whether these indications were generated automatically or manually.

A preliminary CT image may dispensed with; that is, the 3D C-arm image is made in the context of the procedure and is thus automatically recorded and registered with the fluoroscopic images used to guide the catheter and to evaluate the results of the treatment. The intervention becomes markedly safer and faster when compared comparison with the present standard procedure without 3D support.

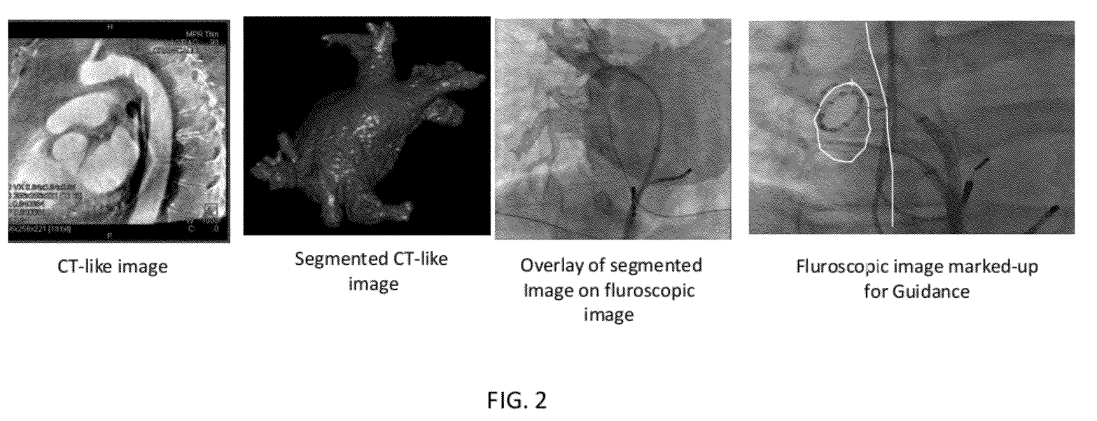

FIG. 2 shows a sequence of images illustrative of the information that is available from the system so as to facilitate the workflow. On the left hand side is a CT-like slice image obtained by the C-arm X-ray device. In the next image, the heart has been segmented from the CT-like data so as to provide a clear picture of the outer surface of the organ. In the second from the right image, the segmented data is processed so as to form an overlay which may be superimposed on the fluoroscopic data in a registered fashion, as the two image data sets were obtained contemporaneously and with the same imaging device. This simplifies the process of registration for superimposition of the images. In the far right image, a fluoroscopic image has been marked up so as to indicate the area of the mitral valve. In addition, the catheter may be clearly seen.

Using the system described above, a method (workflow) for treatment includes the steps of:

1. Obtaining 3D C-arm rotation image data of the heart or left atrium with, or without the administration of contrast agent;

2. Superposition of the 3D C-arm rotation angiography (image data and/or extracted characteristics such as segmentations) as well as lines, measurements, a ruler, and markers, including icons on 2D fluoroscopy;

3. Introducing a guide catheter in the right atrium using fluoroscopy;

4. Puncturing the atrial septum using fluoroscopy with superimposed 3D C-arm rotation image (segmented or soft tissue) for guidance;

5. Piercing the atrial septum and introducing the guide catheter into the left atrium using fluoroscopy with superposition of the 3D C-arm rotation image for guidance;

6. Guiding the MitraClip catheter into the left atrium using fluoroscopy with simultaneous superposition of the 3D C-arm rotation image;

7. Positioning the implantable device (such as the Mira Clip) using fluoroscopy, optionally with simultaneous superposition of the 3D C-arm rotation image, and TEE above or at the cusps of the mitral valve;

8. Firmly clamping the MitraClip to the cusps of the mitral valve;

9. Checking the tightness of the clipped mitral valve in the closed state and its perviousness in the open state, for example, with an acoustic imaging device 66 (see FIG. 1) (If the result is satisfactory, continue with Step 8; if not, return to Step 5);

10. Final clamping of the MitraClip;

11. Removal of all the catheters and tools using fluoroscopy for guidance; and

12. Using fluoroscopy to verify that all the catheters and tools have been removed.

By the superposition of anatomical structures or extracted information, it is possible to perform the workflow steps by using intra-cardial ultrasound and/or extracorporeal ultrasound thus avoiding the use of TEE. The method may be further enhanced by recording the 3D image information using intra-cardial ultrasound (and/or extracorporeal ultrasound), with ensuing superposition on the fluoroscopic images.

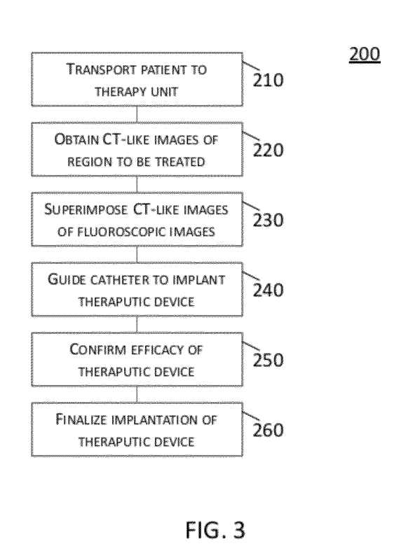

The method may be summarized as a workflow 200 for treating a patient having mitral valve insufficiency, as shown in FIG. 3 including the steps of: transporting the patient to the therapy unit 210; obtaining CT-like image data of the patient region to be treated 220; superimposing CT-like data on fluoroscopic data obtained during the process of catheter treatment of the patient 230, the fluoroscopic data being obtained by the same device as was used to acquire the CT-like image data; using the superimposed data to assist the guidance of the catheter so as to implant a therapeutic device in the patient 240; confirming the efficacy of the therapeutic device 250; if the treatment is effective, and finalizing the implantation so as to permit completion of the procedure 260. If the evaluation of the efficacy of the therapeutic device at step 250 determines that the device has not been effectively placed, the implantation step 240 may be repeated.

While the method disclosed herein have been described and shown with reference to particular steps performed in a particular order, it will be understood that these steps may be combined, sub-divided, or reordered to from an equivalent method without departing from the teachings of the present invention.

Although only a few exemplary embodiments of this invention have been described in detail above, those skilled in the art will readily appreciate that many modifications are possible in the exemplary embodiments without materially departing from the novel teachings and advantages of the invention. Accordingly, all such modifications are intended to be included within the scope of this invention as defined in the following claims.

What is claimed is:

1. A treatment suite for catheterization of a patient, comprising:
a C-arm X-ray device configured to obtain digital image data suitable for producing computed-tomographic-like and fluoroscopic images; and
a catheter device configured to emplace an implantable device in the patient;
wherein a computed-tomographic-image is superimposed on a fluoroscopic image during the use of the catheter device.

2. The apparatus of claim 1, further comprising an acoustic imaging device that is one of non-invasive or minimally invasive, the imaging device configured to obtain images including the implantable device.

3. The apparatus of claim 1, wherein the digital image data used for computed-tomographic-like images is obtained with the administration of a contrast agent to the patient.

4. The apparatus of claim 3, wherein the computed tomographic-like images are segmented to isolate an organ or a portion thereof.

5. The apparatus of claim 4, wherein the organ is the human heart, or portion thereof, including at least one of a left atrium, a right atrium, Chordae, a ventricle, or a valve.

6. A method of treating a patient, the method comprising:
transporting the patient to the therapy unit;
obtaining a computed-tomography (CT)-like image data of the patient region to be treated using a C-arm X-ray device;
superimposing the CT-like image data on fluoroscopic data obtained during the process of catheter treatment of the patient, the fluoroscopic data being obtained by the same device as was used to acquire the CT-like image data;
using the superimposed data to assist the guidance of the catheter so as to implant a therapeutic device in the patient;
confirming the efficacy of the therapeutic device; and
finalizing the implantation of the therapeutic device if an evaluation of the performance device is determined to be satisfactory.

7. The method of claim 6, wherein the step of obtaining a computed-tomography (CT)-like image data of the patient region to be treated includes the step of administering a contrast agent to the patient.

8. The method of claim 6, wherein confirming the efficacy of the therapeutic device includes the use of one of a non-invasive or minimally invasive acoustic imaging device.
